(19) United States
(12) Patent Application Publication
(10) Pub. No.: US 2009/0269858 A1
(43) Pub. Date: Oct. 29, 2009

(54) METHOD OF DETERMINING THE CONCENTRATION OF AN ANALYTE USING ANALYTE SENSOR MOLECULES COUPLED TO A POROUS MEMBRANE

(75) Inventors: Gardiye Hewa Pattinige Kusalini Chamindie Punyadeera, Eindhoven (NL); Hendrik Roelof Stapert, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,769

(22) PCT Filed: Aug. 1, 2007
(86) PCT No.: PCT/IB07/53031

§ 371 Date: Jan. 30, 2009

(30) Foreign Application Priority Data
Aug. 2, 2006 (EP) 061183224

(51) Int. Cl. G01N 33/548; G01N 33/545

(52) U.S. Cl. 436/530; 436/531

(57) ABSTRACT

The present invention relates to a method of determining the concentration of an analyte in a sample and/or the binding kinetics of an analyte to an analyte sensor molecule. For this purpose the invention relies on detecting the interaction between the analyte and the analyte sensor molecule with the latter being physically adsorbed to a solid, porous support which is in the form of a micro array. In a preferred embodiment, the method may be operated in repeated cycles.

### FIELD OF THE INVENTION

The invention relates to a method of determining the concentration of an analyte in a sample which relies on detection of complexes between the analyte and an analyte sensor molecule with the latter being attached to a solid porous support.

The invention further relates to a method of determining real-time binding kinetics of an analyte to an analyte sensor molecule by determining the binding efficiency of the analyte to an analyte sensor molecule with the latter being attached to a solid porous support.

### BACKGROUND OF THE INVENTION

Detection of molecular interactions constitutes one of the core elements in a number of diagnostic tests as well as for general testing procedures. Thus, the presence of a specific analyte in a sample comprising numerous components is usually determined by detecting an interaction between the analyte and an analyte sensor molecule that is known to be specific for this analyte only.

At the same time there is a strong interest in high throughput testing assays, which allow for detection of numerous analytes within a sample in parallel.

Such a high throughput testing may be possible using so-called micro arrays which are miniature detection devices that have been used in various chemical and biochemical applications.

Originally, micro array technology has been developed for detection of specific nucleic acid-nucleic acid interactions. The preference for using micro arrays for detecting nucleic acid based interactions is explained by the fact that nucleic acids are far easier to handle than e.g. proteins and antibodies.

However, in the meantime micro array formats have been developed that also aim at allowing high throughput parallel testing of protein-protein interactions or protein-DNA interactions.

In most micro array-based test systems, typically analyte sensor molecules (capture molecules) are immobilized on the array in defined locations and subsequently the array is incubated with the sample that comprises the analyte(s) to be detected. After certain processing steps an interaction between the analyte and the respective analyte sensor molecule is visualized by e.g. using fluorescent markers.

Typically, micro array substrates use glass cover slides as they allow easy immobilization of nucleotide sequences. However, glass cover slides as well as other solid substrates are not ideal for protein immobilization in view of the complex three-dimensional confirmations of proteins as well as their varying hydrophobic or hydrophilic characteristics. The glass cover slides usually have been coated such that they allow for immobilization of nucleic acids and/or proteins.

Furthermore, efficient interaction between analyte sensor proteins and analytes has been hampered for traditional micro array substrates in that binding has mainly relied on diffusion within a solution to a respective spot of an array. In order to solve these problems more recently so-called flow-through micro array chips have been developed.

In this approach, DNA or protein based probes are immobilized on e.g. a chemically modified porous silicon wafer and then incubated with the sample. Due to the porous nature of the substrate, excess sample as well as optional washing solutions may be removed more easily. An example of such flow-through devices is described in e.g. international patent application WO 03/005013.

Still, the prior art mainly describes methods of detecting the presence of an analyte in a sample as such and does not draw any attention to determining e.g. the concentration of the analyte in a sample or the binding kinetics of an analyte to the analyte sensor molecule.

### OBJECT AND SUMMARY OF THE INVENTION

It is an object of the present invention to provide a method of determining the concentration of at least one analyte in a sample to be tested.

It is also an object of the present invention to provide a method, which allows to determine real-time binding kinetics between an analyte in a sample to an analyte sensor molecule.

In order to achieve the above-defined objects, a method of detection as defined in independent claim 1 is provided.

According to one exemplary embodiment of the present invention, a method of detecting at least one analyte in at least one sample is provided, which method comprises the steps of

a) providing at least one solid porous support with at least one analyte sensor molecule being disposed thereon;
b) contacting said support with at least one sample comprising at least one analyte
c) optionally washing said at least one support with a solution that is capable of removing sample components which have bound non-specifically to said support and/or said analyte sensor molecule;
d) detecting a specific interaction between said at least one analyte sensor molecule and at least one analyte;
e) determining the concentration of said at least one analyte in said at least one sample.

In another exemplary embodiment of the present invention, the concentration may be determined over time. In the latter embodiment the development of the signal over time, which is generated when detecting the specific interaction between the analyte and the analyte sensor molecule, may be used to measure the real-time kinetics of binding of said analyte to said analyte sensor molecule. Depending on the nature of the analyte and the analyte sensor molecules, this embodiment may allow to e.g. identify and characterize the binding of small molecules of a compound library to a therapeutic protein-based target.

In one of the preferred embodiments of the invention, the solid porous substrates may be a membrane selected from the group of membranes that are made of e.g. nylon, nitrocellulose, PVDF or polyethersulfone.

A particularly interesting aspect of the present invention relates to an embodiment wherein the aforementioned steps b) to d) or any of these steps are repeated for multiple times. This repetitive operation of the above-described method has inter alia the advantage that the concentration of the analyte molecules can be determined faster and with higher reliability than with previously known procedures. The same applies if this embodiment of the invention is used in a repetitive manner to determine real-time kinetics of binding of analyte to analyte sensor molecule.

Yet another embodiment of the present invention relates to performing the method with a solid porous substrate as described above in which a multitude of analyte sensor molecules are disposed on said porous substrate in the form of a micro array. The ordered positioning of known analyte sensor molecules on the solid porous substrate and the subsequent incubation with a sample allows determination of the concentration of numerous different analytes in parallel further contributing to the speed and efficiency of the inventive methods. Again, the same holds for determining real time kinetics. This embodiment of the invention may also be operated in a repetitive manner by repeating steps b) to d) multiple times.

In certain preferred embodiments of the present invention, the analyte sensor molecules will be protein-based compounds such as antibodies, peptides, haptens, aptamers, proteins or (cell surface) receptors.

In yet another embodiment of the present invention, the method may be performed with a detectable marker being linked to the at least one analyte and/or to the at least one analyte sensor molecule in order to detect the interaction between the analyte and the analyte sensor molecule.

If the detectable marker is e.g. a fluorescent marker, this may again contribute to easiness, effectiveness and speed of the claimed methods for determining the concentration and/or real-time binding kinetics of an analyte in a sample.

The analyte sensor molecules in one embodiment of the present invention may be covalently attached to the support by way of chemical linkers.

### BRIEF DESCRIPTION OF THE DRAWINGS

FIG. 1 shows a schematic print-request layout for printing antibodies as analyte sensor molecules. A number of different antibodies are printed on the nitrocellulose membrane. The antibodies are allowed to adsorb onto the membrane by physical adsorption. Spots that contain Cy5 labeled antibodies serve as internal calibration standards and serve to anchor the grid.

FIG. 2 shows a measurement of the calibration standards of the micro array depicted schematically in FIG. 1. An image is taken just after printing. Two variables of the software can be fine-tuned to get optimal images i.e. current and the flash length. In this case the current was varied. At a current of 50 mA the low concentration of the internal calibration is not that visible indicating a low signal output. However, when the current is increased to 127.5 mA the low concentration of the internal calibration is visible, indicating that this current is useful.

FIG. 3 shows pictures of an experiment in which the micro array as described in FIG. 2 was incubated with a sample comprising 100 nM rabbit-anti-Mouse-Cy5 labeled antibody. The different pictures represent the signals that were obtained after cycling sample over the membrane (1-8 times).

FIG. 4 relates to the same experiment as FIGS. 2 and 3. The different pictures represent the signals that were there after cycles 5, 6, 7 and 8.

FIG. 5 relates to the same experiment as FIGS. 2, 3 and 4. The upper panel shows the signals obtained after cycle 8 without washing of the micro array. The lower panel shows the signals for the same micro arrays after washing with PBS. 7.4, 0.05% Tween 20.

FIG. 6 shows another micro array print lay out which was used in experiment 2 to monitor the binding of the antigens C-reactive Protein (CRP) and tumor necorsis factor a (TNFa).

FIG. 7 shows the micro array of experiment 2 after printing of the capture antibodies.

FIG. 8 shows the measured signal intensities for the detected antigen CRP at the concentration of 100 nM. The number of the cycle refers to the repeated application of “fresh” streptavidine labeled Cy5. “10+w” indicates that a washing step was included after the last cycle.

FIG. 9 shows the results for the control in which no antigen was added.

FIG. 10 shows the signal-to-noise ratio as calculated on the basis of FIG. 8.

FIG. 11 shows another micro array print lay out which was used in experiment 3.

FIG. 12 shows the micro array of experiment 3 after printing of the capture antibodies.

FIG. 13 shows the normalized intensities for CRP and the markers Alexa647 and Cy5.

FIG. 14 shows the normalized intensities for TNFa and the markers Alexa647 and Cy5.

FIG. 15 shows the kinetics of CRP detection in dependence on repeated cycling.

FIG. 16 shows the kinetics of TNFa detection in dependence on repeated cycling.

FIG. 17 shows the detection principle of experiment 4.

FIG. 18 shows another micro array print lay out which was used in experiment 4.

FIG. 19 shows the normalized signal intensities of analyte binding for experiment 4.

### DETAILED DESCRIPTION OF THE INVENTION

The present invention in one embodiment is directed to a method of detecting at least one analyte in at least one sample comprising the steps of:

a) providing at least one solid porous support with at least one analyte sensor molecule being disposed thereon;
b) contacting said support with at least one sample comprising at least one analyte;
c) optionally washing said at least one support with a solution being capable of removing sample components which have bound non-specifically to said support and/or said analyte sensor molecule;
d) detecting a specific interaction between said at least one analyte sensor molecule and at least one analyte;
e) determining the concentration of said at least one analyte in said at least one sample.

In the past, methods of detecting an analyte in a sample have mainly aimed at allowing a decision as to whether a certain compound is present in a sample or not. However, no methods have been disclosed in which an analyte sensor molecule, which is attached to a solid porous support, is contacted with a sample in order to detect a specific interaction between the analyte sensor molecule and an analyte within the sample and to subsequently use the signal that indicates the interaction between the analyte sensor molecule and the analyte to determine the concentration of the analyte in the sample.

Supports which are useful for the present invention are solid and porous. The term “porous” for the purpose of the present invention means that the substrate is sufficiently permeable for a fluid to pass through it. In a preferred embodiment, the substrate is of a porosity to allow fluid to pass through it upon application of low to moderate over-pressure.

Fluids may comprise solutions, dispersions, suspensions, emulsions, bodily fluids such as blood, plasma, urine, etc. Fluids or fluidic samples may also comprise e.g. environmental samples from the seas, lakes, rivers, etc.

In order to allow a fluid to pass through the pores within the membrane, the term “porous” for the purpose of the present invention will thus typically relate to substrates which comprise (average) pores with diameters of 0.1 to 1 μm 0.2 to 0.8 μm and preferably 0.3 to 0.7 μm and 0.4 to 0.6 μm. A particularly preferred pore size is 0.45 μm.

Furthermore, solid, porous substrates in accordance with the invention will not only have pores of the aforementioned functionality and size but typically also display a porosity number, i.e. a ratio between open volume and membrane material of between 20-80%.

Substrates which are useful for the purpose of the present invention are, of course, known to a person skilled in the art who will understand these substrates to be fabricated among other things from polymeric materials, natural or artificial fibers, silicones, filter materials, membranes and composites thereof. The solid porous substrates in accordance with the present invention may not be formed from aluminum oxide.

Of course, solid supports can be fabricated in all shapes and sizes depending on the particular use. Examples include plates, sheets, disks, films, threads, spots etc. Preferred, but not required shapes are those with a flat planar surface such as a membrane, a filter or a microplate that can be preferably handled by an automated diagnostic system.

Preferred embodiments will use porous membranes as a solid support. These membranes can be made of nylon, nitrocellulose, PVDF or polyethersulfone.

Such membranes can be commercially obtained under the trade names Protran, Ultrabind, Immunodyne, Hybond and Biodyne. The Protran membrane, consisting of nylon re-enforced nitrocellulose, which is available from Pall, is particularly preferred.

The dimension may vary, but membranes having a thickness between 0.1 to 15 mm, between 0.2 to 12 mm, between 0.33 to 11 mm, between 0.4 to 10 mm, between 1 to 10 mm, between 4 to 9 mm or around 8 mm and/or a pore size of 0.1 to 1 μm, between 0.2 and 0.8 μm, 0.3 and 0.7 μm and 0.4 to 0.6 μm are preferred in one embodiment. This is also valid if the membrane is e.g. one of the aforementioned negatively charged nylon membranes.

Analyte sensor molecules which are useful in the present invention are molecules that can be disposed on the solid porous support in a functional confirmation meaning that the analyte sensor molecule while being disposed on the substrate retains the potential for specifically interacting with the target structure which is commonly designated as the analyte.

If a sample comprising various analytes is incubated with an analyte sensor molecule, the analyte sensor molecule will preferably interact with the analyte, which it is specific to, and thus lead to a specific interaction that can subsequently be detected.

Analyte sensor molecules, which are useful for the purpose of the present invention, can be proteins, enzymes, receptors, ligands, antigens, haptens, cells, cellular fragments, small molecules, aptamers and antibodies. Analyte sensor molecules may not be nucleic acids.

In a preferred embodiment the analyte sensor molecules of the present invention will be protein-based analyte sensor molecules. Such preferred analyte sensor molecules include proteins, receptors, antibodies etc. In one embodiment, the analyte sensor molecules will be antibodies that are known to specifically bind with the respective analytes being antigens.

Antibodies to be used as analyte sensor molecules may be of any origin, including mouse, human, rat, chick, sheep, goat etc. and comprise all types of antibodies which are commonly known in the art such as monoclonal antibodies, polyclonal antibodies, chimeric antibodies, humanized antibodies, F(ab), camel antibodies, nanobodies etc.

An overview of different types of antibodies which can be used are found inter alia in Harlow and Lane, Antibodies: A Laboratory Manual, Cold Spring Harbor Laboratory Press, 1988 (e.g. page 17). Furthermore, all types of antibody subclasses such as the aforementioned IgA, IgE, IgM, IgG, IgD etc., may be used. Reference in this context is again made to Harlow and Lane (vide supra).

The analyte sensor molecules can be disposed on and/or within at least a part of any of the aforementioned solid porous supports via covalent or non-covalent linkage to the support.

In a preferred embodiment, antibodies are ink-jet printed onto a nitrocellulose membrane and allowed to adsorb physically. The printed membranes are allowed to dry over night. The printed membranes can preferably be blocked with 5% BSA in PBS pH 7.4 for 1 hour at room temperature followed by the assay.

A covalent linkage between the support and the analyte sensor molecules means that a chemical bond is formed. For the purpose of a covalent linkage the solid porous support can be functionalized in order to provide functional chemical groups that allow formation of a chemical linkage between the support and the analyte sensor molecules.

If thus e.g. antibodies are used as an analyte sensor molecule and should be covalently coupled to the membrane, a membrane may be used that provides functional groups such as carboxyl groups, amine groups, hydroxyl groups, sulfhydryl groups etc. These groups may then be cross-linked either directly to the antibodies or use a linker that may be homo or hetero-bifunctional.

Thus, supports can be activated for providing a chemical linkage to the analyte sensor molecules by e.g. coating an inert solid porous substrate with a polymer having e.g. acyl fluoride functionalities. Other covalent attachment chemistries are also applicable but not limited to anhydrides, epoxides, aldehydes, hydrazides, acyl azides, aryl azides, diazo compounds, benzophenones, carbodiimides, imidoesters, isothiocyanates, NHS esters, CNBr, maleimides, tosylates, tresyl chloride, maleic acid anhydrides and carbonyldiimidazole.

In one embodiment, a carbodiimide functionality may be used for establishing a link between the solid, porous support and the analyte sensor molecule such as an antibody.

For this purpose, a negatively charged nylon membrane such as Biodyne C, membrane which comprises COOH groups, can be pretreated with a linker such as EDAC (N-(3-Dimethylaminopropyl)-N′-ethylcarbodiimide hydrochloride). This active intermediate then reacts with the amino group of an analyte sensor molecules such as antibodies.

Linkage of the antibodies or proteins may take place by using low concentration of EDAC.

Typically these concentrations of EDAC will vary between 0 to 25% by weight of EDAC with 0.5 to 10% by weight, 0.5 to 8% by weight, 0.5 to 4% by weight, 0.5 to 2% by weight and particularly 1% by weight being preferred.

A person skilled in the art is well acquainted with the reaction conditions which have to be regarded when bringing the analyte sensor molecules such as antibodies into contact with a solid, porous support and when contacting an analyte sensor molecule such as antibodies with a sample. Typical reaction conditions depend on certain buffers, temperatures, pH conditions etc. However, these conditions will depend on the type of sample, analyte and analyte sensor molecule as well as on the chemical functionalities that are used in the context of covalent linkage of the analyte sensor molecule to the solid support and will usually be known from the literature or provided by e.g. the manufacturer of chemical cross linking agents.

In one embodiment of the present invention, the method may be performed with a support what comprises only one type of analyte sensor molecule being homogeneously distributed on and/or within at least a part of the support.

However, in another embodiment of the present invention which is particularly preferred, the method is operated with a support wherein analyte sensor molecules are distributed on and/or within at least a part of the solid porous substrate in a spatially ordered and separated manner establishing a support pattern that is usually designated as an “array”.

One of the advantages resulting from disposing the analyte sensor molecules on a support in an array form is that one can dispose different types of analyte sensor molecules and correspondingly different in a known orientation and distribution on an array. Such an orientation will allow the parallel detection of multiple analytes within a sample.

Typically, such arrays will be made of spots which represent a specific analyte sensor molecule. As the identity of the analyte sensor molecule in the spot is known, a complex array can be established. Following industry standards, array formats will have a density meaning the number of spots ranges from 10 to 100000, 50 to 50000, 100 to 10000 or is 1000, 2000, 3000, 4000 or 5000.

A person skilled in the art is of course well aware that for a certain spot of an array a defined amount of analyte sensor molecule may be disposed thereon. Thus, it is possible to provide the solid porous supports in accordance with the present invention in the form of a micro array with the spots of the micro array comprising different amounts of the respective analyte sensor molecules. Providing micro arrays with the spots thereof comprising different amounts of various analyte sensor molecules may be particular interesting if the method is to be used for determining the binding of the real time kinetics binding behavior of an analyte to an analyte sensor molecule (see also below).

An array is thus an arrangement of analyte sensor molecules, particularly biological macro molecules such as polypeptides and antibodies in addressable locations on a solid porous substrate. A micro array is an array that is miniaturized so as to require a minimal amount of analyte sensor molecule and sample for evaluation. Within an array each array molecule is addressable in that its location can be reliably and consistently determined within the at least two dimensions of the array surface. Thus, in ordered arrays the location of each analyte sensor molecule is assigned to the analyte sensor molecule at the time when it is spotted on the array surface and usually a key is provided in order to correlate each location. Often ordered arrays are arranged in a symmetrical grid pattern, but samples could be arranged in other patterns (e.g. in radially distributed lines or ordered clusters).

The shape of the application of analysis sensor molecules or “spot” is in essence immaterial to the nature of the invention. Thus, the micro array of analyte sensor molecules refers generally to a localized deposit of analyte-targeting polypeptide and is not limited to a round or substantially round region. For instance, essentially square regions of polypeptide can be used with arrays of this invention as can be regions that are essentially rectangular (such as slot blot application) or triangular, oval or irregular. The size (diameter of a circular area enclosing the entire spot therein) of the spot itself is immaterial to the invention, though it is usually between 0.1 mm to 0.5 mm. The shape of the array itself is also immaterial to the invention, though it is usually substantially flat and may be rectangular or square in general shape.

The preparation of a micro array refers to the arrangement of different groups of analyte sensor molecules in a spot-to-spot (center-to-center spacing) of between about 0.05 mm to 10 mm or more preferably of between about 0.1 mm to 1 mm.

Arrayers also named array spotters useful in the present invention are currently available from different companies. According to the different spotting techniques, they can be classified into contact mode and non-contact mode (ink jet) arrayers. Contact mode arrayers including systems manufactured by Affymetrix, Amersham Pharmacia, BioRobotics and GeneMachine for example use pen tips that dispense the sample when the tips touch the substrate. The non-contact mode arrayer represented by BioChip Arrayer manufactured by Packard Bioscience (now Perkin Elmer) employ piezo-crystal controlled tips to dispense the pre-sucked sample at about 400 μm above the substrates.

An advantage of the embodiments of the invention in which an array is used is that miniature support platforms can be developed which permit smaller sample sizes and reaction volumes which can lead to economy of scale and time-savings. In addition, these analyzers can achieve comparable or greater sensitivity than conventional micro-assay formats.

The term “analyte” for the purpose of the present invention relates to molecules that are specifically recognized by the aforementioned analyte sensor molecules. An analyte may thus be selected from the group comprising proteins, antigens, haptens, small molecules, lipids, etc. Most preferred the analyte is a protein or a combination of various proteins.

Contacting the solid porous support as described above upon which analyte sensor molecules are disposed preferably in the form of a micro array with the at least one sample may be done by incubating a sample that comprises analytes with the solid support at typical temperatures and conditions.

As has been set out above, it is envisaged that one optionally may wash the support with a solution which is capable of removing sample components which have not specifically bound to the support and/or the analyte sensor molecule.

It is well known that during analyte detection such as for proteins, non-specific binding of components of the sample to be tested to the detection device may occur. This non-specific binding may occur to the support, and/or the analyte sensor molecule. In one embodiment of the invention one may take account of such non-specific binding by removing non-specifically bound components after they have contacted the detection device with the sample using a so-called washing solution for removing the non-specifically bound components.

Furthermore, the inventors of the present invention have found that in one embodiment of the invention it is desirable to treat the support with a solution or liquid that is capable of reducing and/or removing and/or preventing non-specific binding to the support and/or the analyte sensor molecule. Such solutions for liquids will typically be designated as blocking solutions.

The blocking components which are capable of reducing and/or preventing a non-specific binding of sample components to the aforementioned components of the support will typically depend on the nature and amount of the support and/or the analyte sensor molecule.

Blocking components may comprise detergents such as e.g. SDS, TritonX 100, TritonX 80, NP-40, Tween-80, Tween 20 etc. In another embodiment the blocking components of a blocking solution may be BSA, FSA, HSA, Casein or Fc tails. Of course, combinations of the above mentioned blocking agents can also be used.

The latter blocking components, namely BSA, HAS, FSA and particularly Casein, are preferred.

The blocking solution will typically be applied in the form of a solution or liquid such as a blocking buffer. The further components of such e.g. blocking buffers will be salts, acids etc., depending on the specific use. A typical blocking buffer will be PBS pH 7.4 comprising any of the aforementioned components in a concentration of 1%, 2%, 3%, 4%; 5%, 6%, 7%, 8%, 9%, 10% and up to 15% or 20% by weight of BSA, FSA, HAS or Casein.

If components such as Casein, BSA, HSA are used as blocking components within a blocking solution, their concentration will typically be between 1 and 15%, 2 and 10% and preferably between 5 and 10%. These concentrations are percent by weight.

The point of time of blocking may differ. Thus, a support such as a membrane may be pre-blocked before the analyte sensor molecules such as antibodies are bound to the support. The support may also be blocked after an analyte sensor molecule has already been coupled or disposed on the membrane. This may preferably be envisaged if the analyte sensor antibody is absorbed to a membrane such as the above mentioned nylon membranes.

After the sample and the detection device have been brought into contact to allow a specific interaction between the at least one analyte within the sample and the analyte sensor molecule(s) on the support, one may optionally include the above-mentioned so-called washing step which aims at removing and/or reducing non-specifically bound components of the analyte sample which interact non-specifically with the substrate and/or the analyte sensor molecule. After this washing step or, if the washing step is omitted, detection of a specific interaction between the analyte sensor molecules and the at least one analyte of the sample will take place.

It has been set out above for step d) that a specific interaction between the analyte sensor molecule and the analyte may be detected after the support has been brought into contact with the sample.

There are various means of detecting a specific interaction between an analyte within a sample and an analyte sensor molecule. This will be illustrated with respect to an interaction between an antibody as the analyte sensor molecule and a component of a sample such as a protein or a typical chemical compound being specifically recognized by that antibody. However, these explanations are by no means meant to be limited to these exemplary embodiments of the invention.

Detection of the interaction between analyte sensor molecules such as an antibody and the analyte may occur by modifying the analyte with a detectable marker. Modification of the analyte with a detectable marker may take place before contacting the detection device with a sample, during contacting the detection device with the sample or after a specific interaction between the analyte sensor molecule and the analyte has occurred.

Analytes may be modified with detectable markers by modification with e.g. fluorescent components such as Cy5, Cy3, Texas Red, FITC, Attodye, Cydye, Alexa647 etc., radioactive groups etc. If an interaction between the analyte sensor molecule such as an antibody and the e.g. Cy5 labeled analyte occurs, any other labeled components of the sample may be removed by the washing step and presence of the specific analyte in the sample may be detected by e.g. a fluorescent signal resulting from the interaction between the analyte sensor molecule and the analyte that is retained on the detection device by way of its interaction with the analyte sensor molecule.

Other methods such as an induced silver staining may be used for detection. Other detectable markers rely on enzymatic reactions by which a staining is produced, for example horseradish peroxidase may be coupled to the analytes in a sample and later on a reaction may be initiated by providing the corresponding substrates leading to a staining at the positions where an analyte being modified with horseradish peroxidase has interacted with an analyte sensor molecule.

Such enzymatic linkers may further comprise alkaline phosphatase or chemiluminescent systems. Further examples of detectable markers which are also designated as reporter molecules include but are not limited to dyes, chemiluminescence compounds, metal complexes, magnetic particles, biotin, hapten, radio frequency transmitters, and radio luminescence compounds.

Other methods of detecting an interaction between the analyte and the analyte sensor molecule may also be used. For example, if the analyte sensor molecule is an antibody, an analyte may be bound specifically to this antibody from the sample. If then a washing step is used to remove any unbound or non-specifically bound components of the sample is applied, a further antibody, which is also specific to another portion of the analyte, may be added. This antibody may e.g. be linked to a detectable marker. Such a detectable marker may be a fluorescent marker such as Cy5; however, such a marker may also be e.g. an oligonucleotide of known sequence. If, subsequent to the interaction of the so-called secondary antibody with the analyte being retained on the support by way of the capture antibody, this oilgonucleotide is used for a polymerase chain reaction (PCR), presence of the analyte in the sample may be detected. This so-called immuno PCR is very sensitive and can be used to reliably detect minute amounts of analyte within the solution. The above detection methods which rely on a secondary analyte sensor molecule such as an antibody are also called “sandwich assays”. The skilled person is well acquainted with such assays.

In yet another preferred approach, the secondary (detection antibody) is coupled to e.g. biotin, In a further step the analyte sensor molecule-analyte-secondary antibody complex is contacted with a strepatvidine labeled detectable marker such as a fluorescent marker. The fluorescent marker can then interact via streptavidine with the biotin-labeled secondary antibody.

There are of course other alternatives to this approach possible. For example, an analyte sensor molecule such as a capture antibody may be printed on a porous membrane by means of an ink jet printer. Subsequently, the printed membrane can be blocked to minimize unspecific binding. A sample containing e.g. target proteins being the analyte in this case will then be directly labeled. The label may be a fluorophor, a gold bead, an enzyme or a hapten such as described above. Using the below described flow-through set up, the labeled sample will be pumped a couple of times across the membrane to allow antibody-antigen binding. Excess label will be removed by an additional optional washing step.

In yet another embodiment, the analytes which may be target proteins are printed onto a porous membrane by using an ink jet printer. The printed membrane will be blocked to minimize unspecific binding and a sample containing also target proteins is then mixed with several optionally labeled antibodies that can bind to the analyte. The label can e.g. be a fluorophor. Optionally, washing steps are then applied to remove unspecifically bound antibodies or labels from the membrane. Also optionally, an incubation step with the second label antibody that targets bound antibodies can be carried out. The aforementioned optional alternatives can also be combined. The antigen-antibody 1 or antigen-antibody 1-antibody 2 binding will then be visualized by a detection set up which is e.g. capable of detecting fluorescent signals and quantification of the complex will be carried out using calibration standards (see below). The latter embodiment is actually a competition assay. A high concentration of target protein in the input sample will give a low signal on the membrane. Furthermore, the amount of the first capture antibody should be carefully optimized not to be in excess compared to the number of protein targets in the sample.

Of course, a specific interaction between an analyte sensor molecule and an analyte may also be detected without a marker, i.e. so-called “label-free” detection. This may e.g. be done with the Biacore system.

If, as mentioned above, analyte sensor molecules are disposed on the support in an array format, the method in accordance with the invention may be used to allow the parallel processing of samples and parallel detection of numerous analytes within one or multiple samples. As each detection spot may correspond to a different analyte sensor molecule, which is specific to a certain analyte, a signal originating from such a detection spot after the solid porous support has been incubated with a sample and processed in the above-described manner will be indicative of the presence of an analyte within a sample.

In the last step e) in the methods in accordance with the present invention the concentration of the analyte in the sample is determined.

Determination of the concentration mainly relies on the signal that is generated upon detection of the specific interaction between the analyte sensor molecule and the analyte as described above for step d).

Typically, one will first contact a support on which analyte sensor molecules have been disposed, on with a sample comprising an analyte is capable of specifically binding to the analyte sensor molecule on the support, with the concentration of the analyte sensor molecule being known. By using samples with known, but different concentrations for this specific analyte and e.g. using a micro array with spots of different amounts of an analyte sensor molecule and a fluorescent marker that is e.g. coupled to the analyte, one will then establish a so-called calibration or standard curve in which a certain fluorescent signal intensity is correlated with the known concentration of the analyte within the sample.

In a second step which is the actual measurement, a sample is taken comprising analytes with the concentration thereof being unknown. These samples are processed in a method in accordance with the invention as described and if e.g. the analyte is again labeled with a fluorescent marker of the same identity as has been used for establishing the calibration curve, a fluorescent signal is recorded. This fluorescent signal can then be correlated with a concentration of the sample by comparison with the aforementioned calibration curve.

The person skilled in the art will of course be aware that fluorescent signals are subject to saturation effects which e.g. occur if a sample is brought into contact with the support with the sample comprising more analyte than analyte sensor molecules being available for interaction on the support. In such cases one will dilute stepwise the analyte sample until one has reached a situation where a non-saturated fluorescent signal is achieved.

Other embodiments of determining the concentration of an analyte in a sample will be known to the person skilled in the art.

If, for example, a chemical or enzymatic reaction is used to detect an interaction between the analyte and the analyte sensor molecule by way of a calorimetric reaction again, a calibration or standard curve may be established with samples for which the concentration of the analyte is known. Subsequently, the method as described above in accordance with the invention will be performed and from the obtained colorimetric value a concentration will be calculated. The person skilled in the art will again be aware that one will have to ensure that only such colorimetric signals are considered that have not yet reached the saturation level.

One of the advantages of the method as described above for determining the concentration of an analyte within a sample is that the sample is brought into contact with a porous support. As the support is porous, the sample can pass through the support, which allows to fast and efficiently remove all non-specifically bound components of the sample leading to an overall enhanced signal-to-noise ratio once the specific interaction between the analyte and the analyte sensor molecule has been detected.

Furthermore, the use of a porous membrane allows an advantageous and preferred embodiment of the invention namely that the above method is performed in repetitive manner by repeating aforementioned steps b) to d) multiple times.

Preferably steps b) to d) are repeated at least 2, 3, 4, 5, 6, 7, 8, 9, 10 and up to 30 times. A particularly preferred cycle time is approximately 8 to 12 cycles. The repetitive contacting, optional washing and detection of the specific interaction between the analyte sensor molecule and the analyte may be facilitated by e.g. pumping the sample over the solid porous support in cycles. Cycling may also be facilitated by applying a vacuum in addition to or as an alternative for pumping. Of course, the person skilled in the art will know how to adapt an aperture for constantly pumping the sample over the support if e.g. a washing step is to be included. This may e.g. be done by using a line that can be switched by a valve to different sources such as sample and washing buffer source.

The repetitive application of the sample over the support leads to a significantly enhanced signal-to-noise ratio when detecting the interaction between the analyte sensor molecule and the analyte. This is particularly true when the repetitive cycling is interrupted by contacting or incubation periods of e.g. at least 20 seconds, at least 30 seconds, at least 40 seconds, at least 50 seconds, at least 60 seconds, at least 2 minutes, at least 3 minutes or at least 5 minutes. The number of cycles as specified above may be applied. The repetitive application of sample may include the repetitive addition of fresh sample and/or sample that has already been passed once over the membrane, i.e. the “flow-through” of the first passage.

The term “repetitive cycling” may not only refer to repeating all steps b) to d) altogether. Instead, the term “repetitive cycling” may also refer to any of these steps b) to d) or even substeps thereof. For example, instead of cycling the sample numerous times over the porous membrane, one may also only repeatedly apply, i.e. cycle a washing solution over the membrane.

Similarly, one may repeatedly cycle the detectable marker over the support. This may e.g. be preferably the case if a streptavidine-labeled fluorescent marker is used to detect a biotin-labeled secondary antibody.

Of course, a combination of the aforementioned cycling steps, i.e. cycling of sample, washing solution and/or detectable marker may also be used.

Without being wanted to be bound to a theory, it is assumed that the repetitive cycling which is preferably interrupted by the aforementioned incubation periods increases signal-to-noise ratio when detecting the interaction between the analyte sensor and analyte because of the repeated on- and off-reactions between analyte and analyte sensor molecules which leads to efficient removal of non-specifically bound components of the sample and favors a selective pressure for specific interactions.

Furthermore, the repetitive contacting of the sample with the support seems to be responsible for the fact that the overall incubation period being required for an efficient detection is significantly reduced compared to the situation where a method is not performed in repetitive cycles but where only one incubation and optional washing step is included.

Thus, the present invention, when being performed in the above-described repetitive manner, has the advantage that the time required to e.g. detect an immunological reaction between an antibody and its antigen can be reduced from conventional duration, which may last for up to 3.5 hours down to 15 minutes if e.g. no incubation on the membrane is performed. If incubation steps are included, the total assay time for 5 pump cycles can be ˜45 minutes. Repetitive operation of the method in accordance with the invention may be achieved by various means with pumping and/or vacuum means being preferred as they allow a controllable and constant flow of the sample and optional washing solutions over and through the support.

Another embodiment of the present invention relates to the situation where the concentration of the analyte in the sample is determined over time.

Determining the bound fraction over time allows to measure the real-time kinetics of binding of an analyte to its analyte sensor molecules. When determining real time kinetics of binding of an analyte to an analyte sensor molecule one can convert the signal into the concentration of the analyte. Depending on the type of association curve, one needs to know the association rate constant(s), dissociation rate constant and the analyte sensor molecule density (i.e. number of analyte sensor molecules per surface area). Association and dissociation constants can be determined separately. The capture molecule density can be determined from the concentration and volumes deposited and the diameter of a spot. Alternatively, one may determine association or dissociation rate constants when incubating with analyte molecules of known concentrations. For detecting the specific interaction one may again rely on the aforementioned detectable markers such as fluorescent molecules.

Once one has established a binding curve that reflects the kinetics of binding of an analyte to an analyte sensor molecule, a sample comprising an analyte of unknown concentration may be contacted with the solid support and detection of the specific interaction between analyte and analyte sensor molecule may again be monitored over time. By determining the curve shape for the sample of unknown concentration with the different standard curves that have been obtained for the samples of different but known concentration one can estimate not only the binding kinetics parameters of the binding of the analyte to the analyte sensor molecule, but also the concentration of the analyte within the sample.

Determination of real-time kinetics of binding of analyte to analyte sensor molecule may be significantly improved and time required for measuring be reduced if in steps b) to d) or any of these steps are repeated multiple times. In this context, reference is made to the repetitive operation of the method in accordance with the invention as described above. Of course, the repetitive operation of the method may be interrupted by incubation and contacting periods as described above.

There are multiple useful applications for the present invention. Thus, the above-described method may be used for immunological reactions which detect a certain antigen in e.g. blood samples. Thus, the above-described methods may be used for diagnostic purposes. A particular advantage of the above-described method is that it not only allows to detect the presence of a certain analyte in a sample but also to determine the concentration thereof in a comparatively short time span.

Moreover, the above-described method can be used to determine the real-time kinetics of binding of an analyte to an analyte sensor molecule. The above-described method may thus be used not only to detect e.g. a small molecule compound in a compound library, which is capable of interacting with a therapeutically interesting target such as e.g. a cellular receptor, but also to determine the binding kinetics of the interaction between the small molecule and its target protein.

Thus, the present invention may be used to determine the presence and concentration of an analyte within a sample with the sample being retrieved from different sources such as environmental sources, blood, lymph, etc.

Additionally and/or alternatively, by relying on the embodiments of the invention that are concerned with determining the real time kinetics of an interaction between analyte sensor and analyte molecule, the inventive method may be used to determine the presence and binding behavior of an analyte in a sample with respect to an analyte sensor molecule.

In an alternative embodiment of the invention, the method of detecting at least one analyte in at least one sample may comprise the steps of

a) providing at least one solid porous support with at least one analyte sensor molecule being disposed thereon;
b) contacting said support with at least one sample comprising at least one analyte;
c) optionally washing said at least one support with a solution that is capable of removing sample components which have non-specifically bound to said support and/or said analyte receptor molecule;
d) detecting a specific interaction between said at least one analyte sensor molecule and at least one analyte;
e) determining the real time binding kinetics of the analyte to the analyte sensor molecule.

If e.g. steps b) to d) (or any of these) are again repeated for multiple times as described above and if the optional incubation periods are kept sufficiently short, an analyte being present in a sample may not immediately react completely with the analyte sensor molecule that may be present in excess.

In this situation, i.e. when the method is operated in a repetitive manner the signal detecting a specific interaction between the analyte and analyte sensor molecule may be recorded after each cycle over time. As more and more analyte will bind to the analyte sensor molecule upon repeated cycling, one will observe an increase in signal intensity that will level out once binding of the analyte to the analyte sensor molecule has reached saturation. If one now performs the method in this repetitive manner and applies different samples each with a different known concentration of a specific analyte, one can calculate calibration or standard curves that reflect the binding kinetics at different amounts of analyte to analyte sensor molecules over time.

If one now repeats the method with a sample containing an analyte of unknown concentration under identical conditions, i.e. the same number of repetitive cycles and optional incubation and washing periods, one will also obtain a signal against a time curve that reflects the binding behavior of the analyte to the analyte sensor molecule over time. Thus, it will be possible to monitor the binding kinetics of an analyte in a sample of unknown concentration and to determine the concentration of the analyte simultaneously.

In the following, the invention is illustrated in view of certain experimental examples. These examples are however in no way meant to limit the invention as to its scopes, but rather serve to illustrate the invention by way of some of its exemplary embodiments.

### Experiments

### Experiment 1

### Preparation of a Membrane of Printing Antibodies

In order to obtain a solid porous support in a micro array format, a nitrocellulose membrane was used. Different antibodies were disposed on this membrane using an ink jet printer.

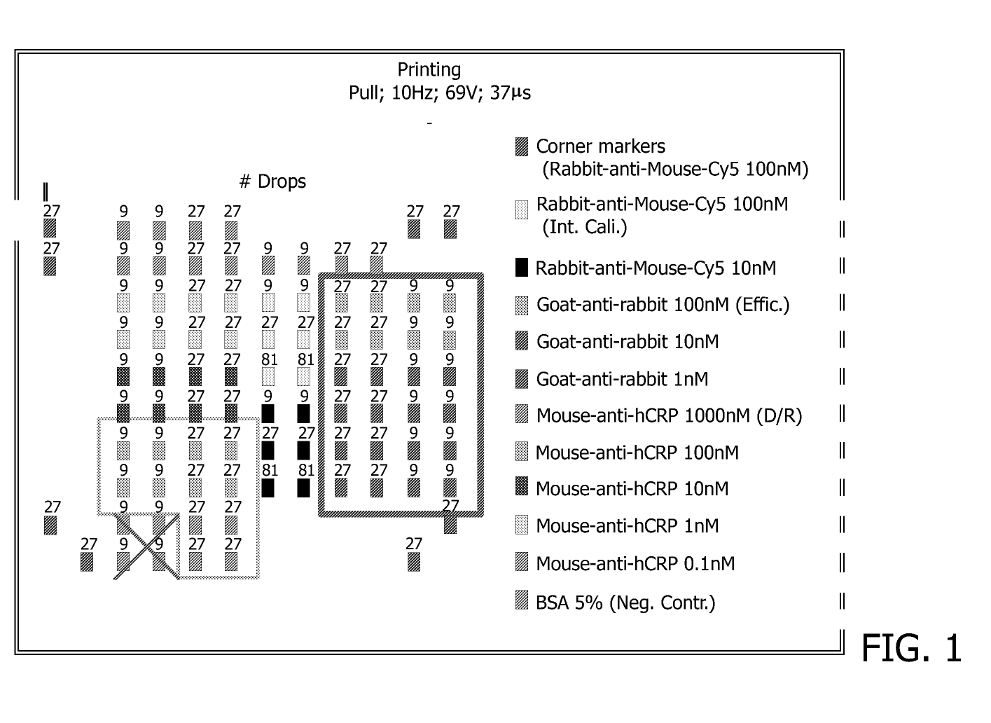

In FIG. 1, a schematic picture of the micro array obtained is shown. Antibodies which comprise a fluorescence label such as Cy5 will lead to a fluorescent signal upon suitable excitation regardless of whether an analyte is bound or not. These analyte sensor molecules serve to identify the edges and corners of the micro array and to provide internal standards for calibration of the detection device that is used to measure the resulting fluorescence.

### Detecting Analytes on a Micro Array

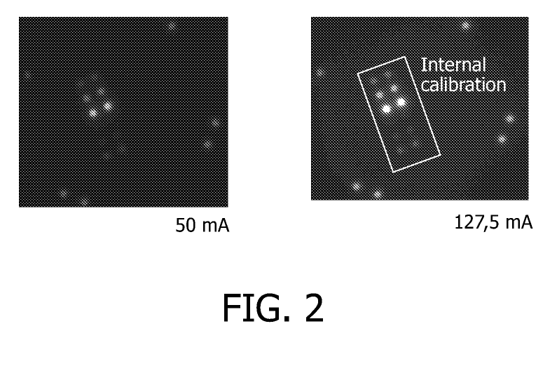

In the following, the afore-described micro array was then blocked overnight with 5% by weight BSA, which was protease-free in PBS pH 7.4. Subsequently, the pictures depicted in FIG. 2 were taken. Values of 127.5 mA and 50 mA indicate the images taken at different currents to drive the LEDs of the optical system.

In a second step 300 μl of 100 nM Rabbit-anti-Mouse Cy5 labeled antibody was added to the micro array in the first chamber. The micro array was incubated with the sample for about a minute. Subsequently, the sample was pumped various times over the micro array without intermediate washing steps. After each incubation step, the interaction between the analytes in the sample, i.e. the Rabbit-anti-Mouse Cy5 labeled antibody with the analyte sensor molecule, i.e. the Goat-anti-Rabbit antibody was monitored taking pictures after excitation with a Philips developed LED-setup (wavelength ex/em:650/670 nm). The incubation time was approximately 1 minute for each cycle.

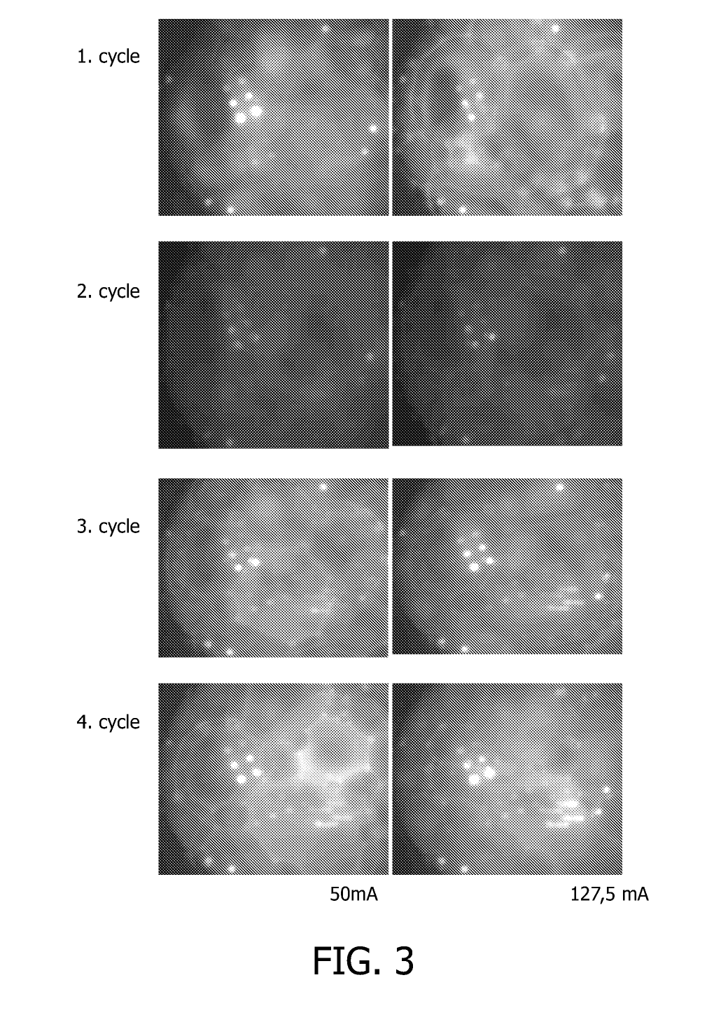

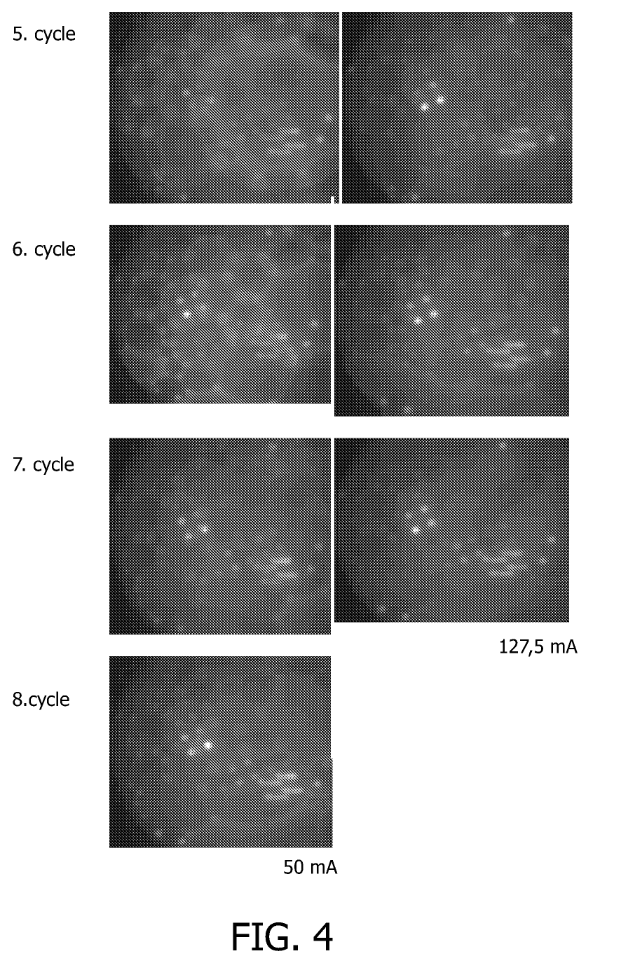

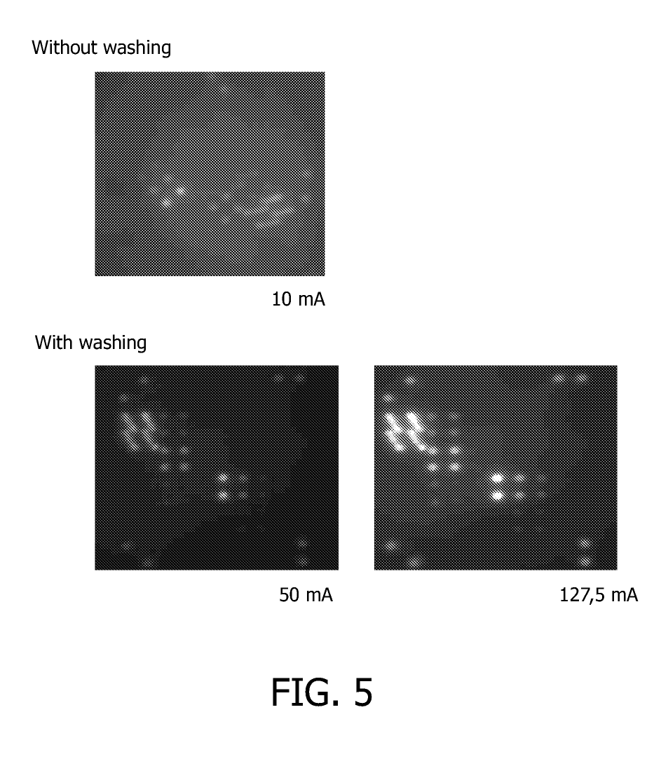

FIG. 3 shows the pictures after cycles 1, 2 and 4. FIG. 4 shows the recorded pictures after cycles 5, 6, 7 and 8. The lower panel of FIG. 5 shows the results obtained if one washes after the last cycle (cycle 8) with the micro array three times with PBS pH 7.4, 0.05% by weight Tween 20. The upper panel of FIG. 5 shows the same picture without washing.

From the last picture one can clearly see that an interaction between the Rabbit-anti-Mouse Cy5 labeled antibody and the Goat-anti-Rabbit antibodies has taken place. Furthermore, one can see clearly stronger signals in the case where more Goat-anti-Rabbit antibody has been disposed on the micro array than in the case where lower amounts have been disposed on the micro array.

### Experiment 2

In this example the influence of repeated cycling of the detectable marker on the signal strength was tested.

### Micro Array Print Lay Out

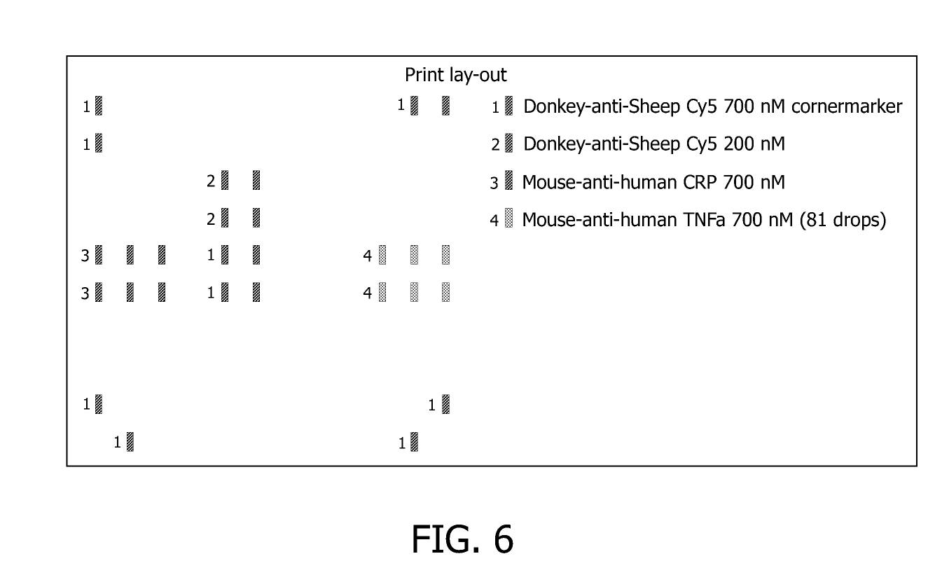

A micro array print lay out as shown in FIG. 6 was used. The micro array was produced as described above. Cy5 labeled antibodies again represent internal standards.

### Experimental Set Up:

The following capture antibodies (analyte sensor molecules), antigens (analytes) and secondary detection antibodies were used. Streptavidine-labeled Cy5 was used for detection. PBS always refers to PBS pH 7.4. PBST always refers to PBS pH 7.4, 0.0% Tween 20.

| Name | Conc. | Article nr | Company |
|---|---|---|---|
| CRP capture antibody | 9 mg/ml | 4C28 | Hytest Ltd. |
| CRP-9 antigen | 2.8 mg/ml | 1707-2004 | Biotrend |
| CRP detection antibody | 1.7 mg/ml | 4C28B | Hytest Ltd. |
| TNFα capture Ab | 0.5 mg/ml | 14-7348-81 | eBioscience |
| TNFα human recombinant | 0.1 mg/ml | 14-8329-63 | eBioscience |
| TNFα detection Ab | 0.5 mg/ml | 13-7349-81 | eBioscience |

The components were used in the following concentrations:

### Capture Antibody

### Mouse-Anti-Human CRP

700 nM 3.5 μl stock+296.5 μl PBS with 5% glycerol

### Mouse-Anti-Human TNFα

700 nM 63 μl stock+237 μl PBS with 5% glycerol

### Donkey-Anti-Sheep Cy5 Labeled

700 nM 21 μl stock+279 μl PBS with 5% glycerol
200 nM 6 μl stock+294 μl PBS with 5% glycerol

### Antigen

### CRP/TNFα Antigen

100 nM 8.2 μl stock CRP+34 μl TNFα+1957.8 μl PBS with 1% BSA p.f.
100 pM 2 μl of 100 nM solution+1998 μl PBS with 1% BSA p.f.
Blank PBS with 1% BSA p.f

### Secondary Detection Antibody

### Mouse-Anti-Human CRP/Mouse-Anti-Human TNFα

### 66.7 nM CRP+6.67 nM TNFα 58.8 μl CRP+20 μl TNFα+9921.2 μl PBS

Detection antibodies were all labeled with biotin.

### Detection System

### Streptavidin Cy5 Labeled

1:1000 10 μl stock+9990 μl PBS

The procedure for incubating the micro array and detecting the interactions was as follows. The experiments were performed in duplicate:
print array on membrane
take an image of the array
block the arrays with 1000 μl PBS+5% BSA for 1 hr (room temperature (RT), dark)
take an image of the array to determine recovery
add 500 μl antigen solution to the array
open the vacuum and wait until the fluid is completely removed
repeat this 4 more times
wash the array by pipetting 500 μl PBS
open the vacuum and wait until the fluid is completely removed
repeat this 2 more times
add 500 μl detection antibody solution to the array
open the vacuum and wait until the fluid is completely removed
repeat this 4 more times
wash the arrays 3 times with PBS-T as before
take an image of the array
add 500 μl Strep-Cy5 solution to the array
open the vacuum and wait until the fluid is completely removed
take an image of the all array
repeat the last 3 steps as many times as needed

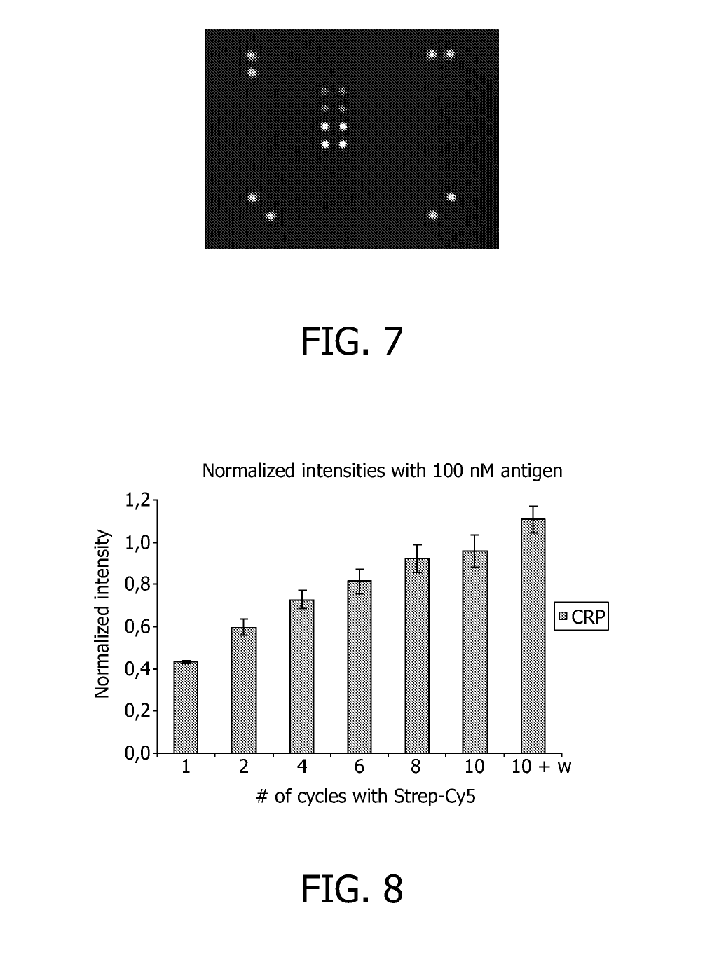

FIG. 7 shows the array just after printing. In the table below the recovery values for the capture antibodies are shown, which are calculated on the basis of the 200 nM and 700 nM donkey-anti-sheep Cy5 labeled internal standards.

| Solution | Before | After | Recovery |
|---|---|---|---|
| 200 nM | 0.0535 | 0.0207 | 39% |
| 700 nM | 0.1774 | 0.0658 | 37% |

| | |
|---|---|
| Average recovery = | 38% |

### Results

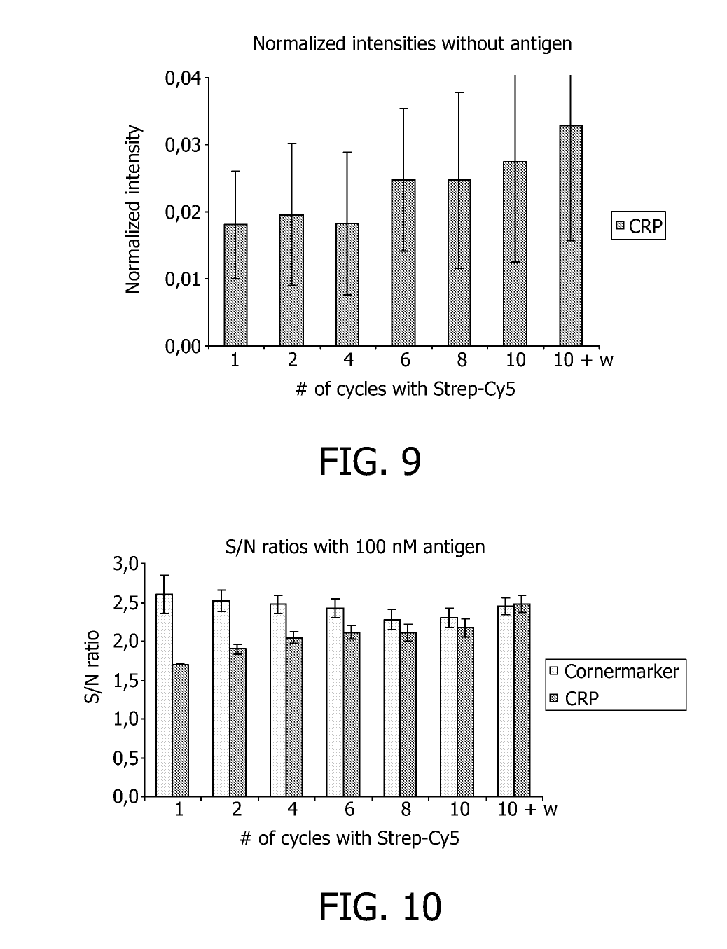

FIGS. 8 and 9 show normalized intensities which were measured after repeated cycling (i.e. 10 times including a final washing step) for bound C-reactive protein (CRP); tumor necrosis factor a (TNFa) at different concentrations as well as for the blank. FIG. 10 shows the signal-to-noise (S/N) ratio for CRP and TNFa as determined on the basis of FIGS. 8 and 9.

The experiment shows that the assay can be efficiently carried out in 15 minutes.

### Experiment 3

In this example the influence of the detectable marker was investigated. Further the effect of pumping was investigated as well as cycling of flow-through through the micro array.

### Micro Array Print Lay Out

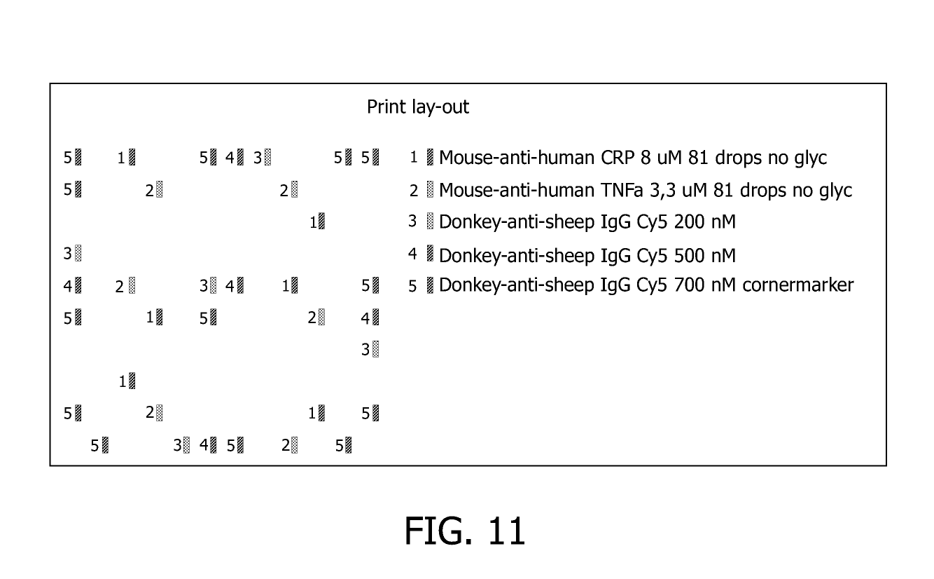

A micro array print lay out as shown in FIG. 11 was used. The micro array was produced as described above. Cy5 labeled antibodies again represent internal standards.

### Experimental Set Up:

The following capture antibodies (analyte sensor molecules), antigens (analytes) and secondary detection antibodies were used. Streptavidine-labeled Cy5 was used for detection. PBS always refers to PBS pH 7.4. PBST always refers to PBS pH 7.4, 0.0% Tween 20.

| Name | Conc | Articlenr | Company |
|---|---|---|---|
| CRP capture antibody | 9 mg/ml | 4C28 | Hytest Ltd. |
| CRP-6 antigen | 2.8 mg/ml | 1707-2004 | Biotrend |
| CRP detection antibody | 1.7 mg/ml | 4C28B | Hytest Ltd. |
| TNFα capture Ab | 0.5 mg/ml | 14-7348-81 | eBioscience |
| TNFα human rec. | 0.1 mg/ml | 14-8329-63 | eBioscience |
| TNFα detection Ab | 0.5 mg/ml | 13-7349-81 | eBioscience |
| Donkey-anti-sheep Cy5 | 1.5 mg/ml | 713-175-003 | Jackson Immuno |

The components were used in the following concentrations

### Capture Antibody

### Mouse-Anti-Human CRP

8 μM 20 μl stock+180 μl PBS

### Mouse-Anti-Human TNFα

3.33 μM Undiluted stock

### Donkey-Anti-Sheep Cy5 Labeled

200 nM 6 μl stock+294 μl PBS with 5% glycerol
500 nM 15 μl stock+285 μl PBS with 5% glycerol
700 nM 21 μl stock+279 μl PBS with 5% glycerol

### Antigen, Secondary Detection Antibody

100 nM 8.2 μl stock CRP+34 μl stock TNFα+1906.1 μl PBS with 1% BSA
100 μM 1.9 μl of above solution+1946.3 μl PBS with 1% BSA
100 fM 1.9 μl of above solution+1946.3 μl PBS with 1% BSA
Blank 1948.2 μl PBS with 1% BSA
Next add to all solutions:
66.7 nM 11.8 μl stock CRP detection antibody
66.7 nM 40 μl stock TNFα detection antibody
Detection antibodies were all labeled with biotin.

### Detection System

1:1000 10 μl stock streptavidin-Cy5+1990 μl PBS
1:1000 10 μl stock streptavidin-A647+1990 μl PBS

The procedure for incubating the micro array and detecting the interactions was as follows. The experiment was performed in duplicate:
print arrays
take an image of arrays
for this experiment nylon membrane arrays are used
block the arrays with 500 μl/well PBS+5% BSA for 1 hr
wash the arrays 3*5 min in PBST and take image to determine the recovery
incubate other arrays with different concentrations of antigen and detection antibody 500 μl for 5 minutes. Then apply vacuum until the membrane is dry.
Next incubate streptavidin-dye for 2 minutes. Then again apply vacuum until the membranes are dry. Take images of all membranes.
Incubate the flow-through for 5 minutes. Afterwards, apply vacuum until the membranes are dry and take images. Repeat this for 3 more steps.
Incubate fresh streptavidin-dye solution for 5 minutes. Afterwards, apply vacuum until the membranes are dry. Take images.
Wash the membranes by pipetting 500 μl PBS-T and applying vacuum until the membranes are dry. Repeat this two times, then take images.
   The cycles may thus be summarized as:
   Cycle 1: 5 min antigen+detection antibody, next 2 min streptavidin-dye
   Cycle 2-4: 5 min flow-through
   Cycle 5: 5 min fresh streptavidin-dye
   Cycle 5+w: 3 times PBS-T (without incubation on membrane)

FIG. 11 shows the array just after printing. In the table below the recovery values for the capture antibodies are shown which are calculated on the basis of the 200 nM, 500 nM and 700 nM donkey-anti-sheep Cy5 labeled internal standards.

| Solution | Before | After | Recovery |
|---|---|---|---|
| 200 nM | 0.0431 | 0.0060 | 13.8% |
| 500 nM | 0.1376 | 0.0224 | 16.3% |
| 700 nM | 0.2090 | 0.0308 | 14.7% |
| | | Av = | 15.0% |

### Results

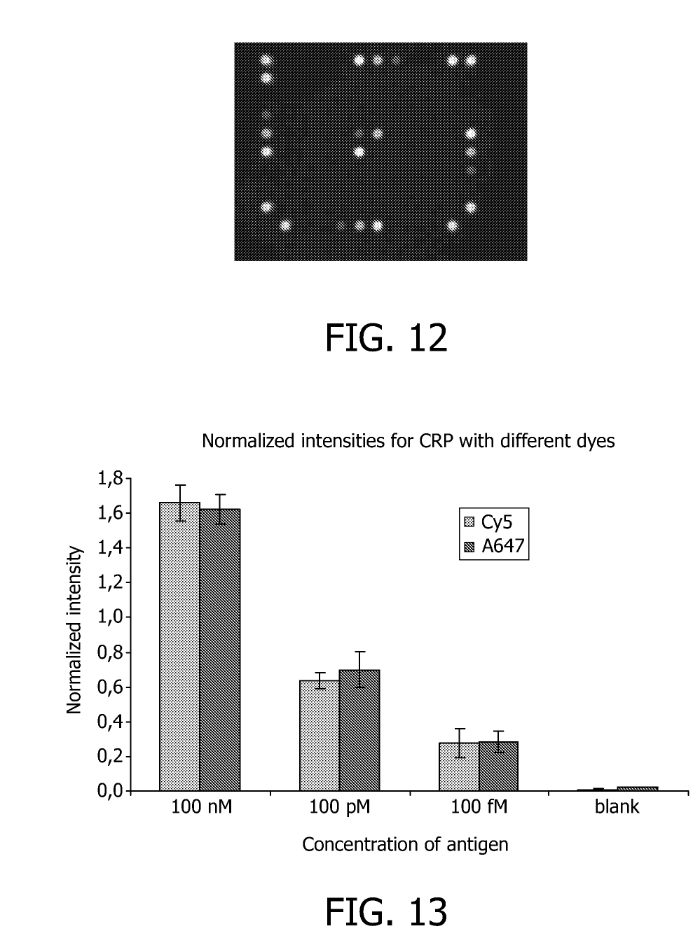

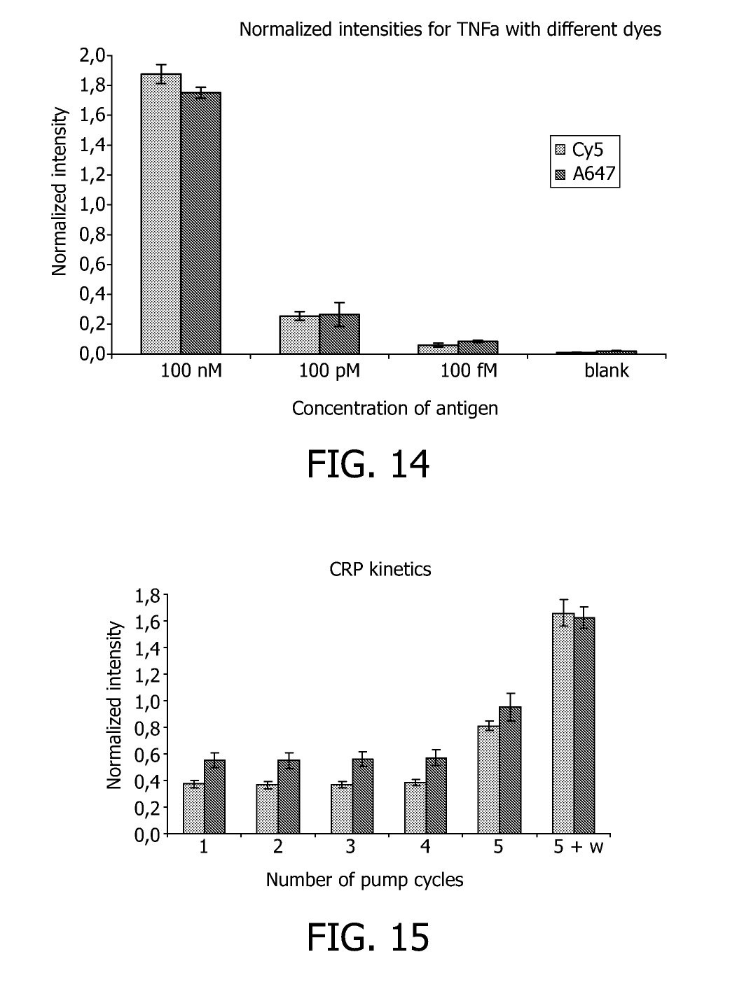

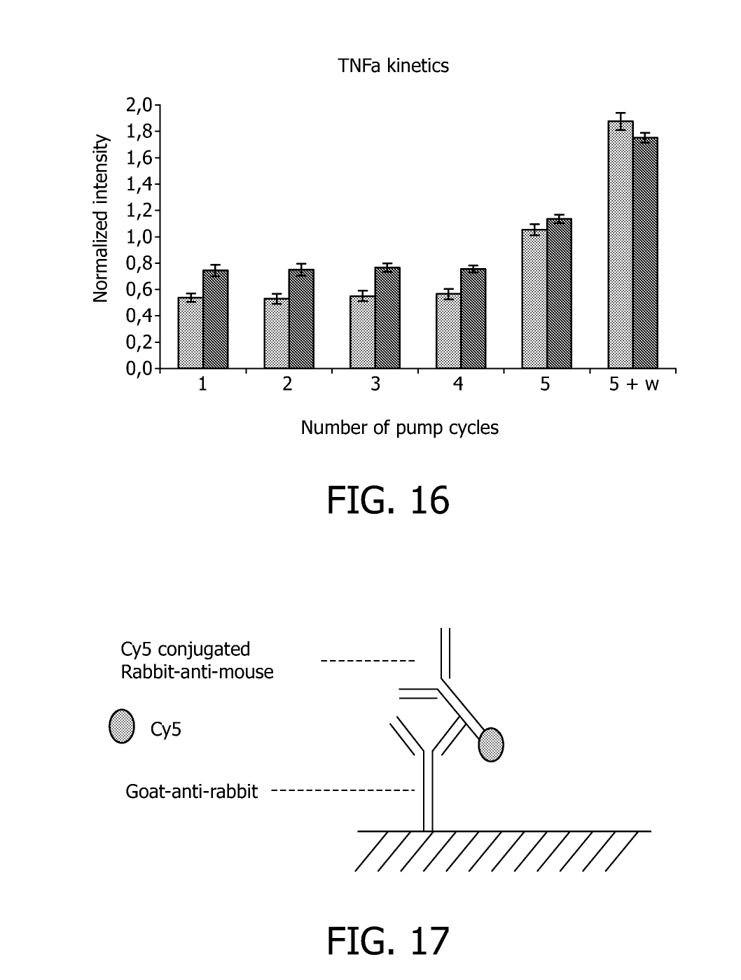

FIG. 12 shows a micro array picture taken after blocking for determining recovery. FIGS. 13 and 14 show the normalized intensities for CRP and TNFa at different concentrations and dyes. FIGS. 15 and 16 show the kinetics of detection of CRP and TNFa in dependence on the various cycles.

The results clearly indicate that the immunoassay can be preformed on a short time scale and that pumping, application of vacuum and repeated operation of certain steps can influence the measured signal intensities.

### Experiment 4

In this example 700 nM of a goat anti rabbit antibody was printed on a nylon membrane. The membrane was incubated with 100 nM of rabbit-anti-mouse-Cy5 (see FIG. 17). For incubation the analyte solution was pumped over the membrane 5 times. The experiment was performed in triplicate.

### Micro Array Print Lay Out

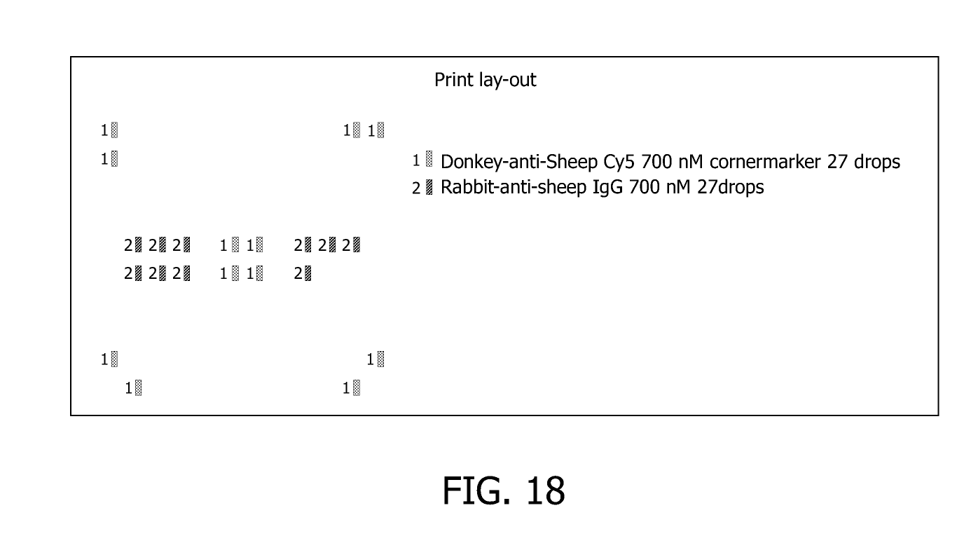

A micro array print lay out as shown in FIG. 18 was used. The micro array was produced as described above. Cy5 labeled antibodies again represent internal standards.

### Results

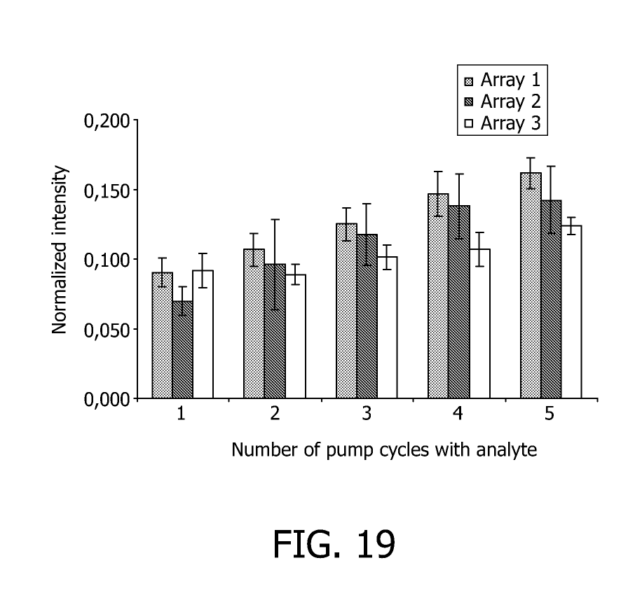

FIG. 19 shows that with repeated cycling of the analyte solution, normalized signal intensities increase.

Three major conclusions can be drawn from the above experiments: First, by rapid cycling of a sample over a micro array it is possible to significantly reduce the time needed for an efficient detection of an interaction between analyte and an analyte sensor molecule. In the above-described experiment 1, the overall time for detecting the signal took approximately 10 minutes if no incubation is carried out on the membrane. This contrast with the time spans that are typically needed in the prior art for such applications that can amount to up to 3.5 hrs.

Second, by comparing the signals obtained in e.g. FIG. 5 with a standard calibration curve that had been established before one may determine the concentration of the Rabbit-anti-Mouse Cy5 labeled antibody. This applies correspondingly to the other experiments

Third, by monitoring the development of the signal strength over each cycle, i.e. over time, one may actually determine the binding kinetics of the analyte which in the case of Experiment 1 would be Rabbit-anti-Mouse Cy5-labeled antibody to its capture antibody given that the capture antibody which in Experiment 1 is Goat-anti-Rabbit antibody has been disposed in different concentrations on the micro array leading to different signal strengths and intensities at the different points of time.

1. Method of detecting at least one analyte in at least one sample, the method comprising the steps of:
a) providing at least one solid porous support with at least one analyte sensor molecule being disposed thereon;
b) contacting said support with at least one sample comprising at least one analyte;
c) optionally washing said at least one support with a solution that is capable of removing sample components which have bound non-specifically to said support and/or said analyte sensor molecule;
d) detecting a specific interaction between said at least one analyte sensor molecule and said at least one analyte;
e) determining the concentration of said at least one analyte in said at least one sample.

2. Method according to claim 1,
wherein the concentration is determined over time to measure the real-time kinetics of binding of said analyte to said analyte sensor molecule.

3. Method according to claim 1,
wherein the porosity of said substrate allows a fluid to flow through it.

4. Method according to claim 3,
wherein the substrate is a membrane selected from the group comprising membranes which are made of nylon, nitrocellulose, PVDF or polyethersulfone.

5. Method according to claim 1,
wherein steps b) to d) or any of these steps are repeated multiple times.

6. Method according to claim 1,
wherein a multitude of analyte sensor molecules are disposed on said support in the form of a micro array.

7. Method according to claim 1,
wherein said at least one analyte sensor molecule is an antibody that is specific to an analyte.

8. Method according to claim 1,
wherein said at least one analyte of said at least one sample is modified with at least one detectable marker.

9. Method according to claim 8,
wherein said at least one detectable marker is selected from the group comprising fluorophores, enzymes, dyes, chemiluminescence compounds, radioisotopes, metal complexes, magnetic particles, biotin, haptens, radio frequency transmitters and radio luminescence compounds.

10. Method according to claim 1 wherein the analyte is a protein.
